(19) United States
(12) Patent Application Publication
(10) Pub. No.: US 2021/0391051 A1
(43) Pub. Date: Dec. 16, 2021

(54) INFORMATION PROCESSING APPARATUS, INFORMATION PROCESSING METHOD, AND PROGRAM

(71) Applicant: SONY CORPORATION, TOKYO (JP)

(72) Inventors: KAZUKI OCHIAI, TOKYO (JP); HIROAKI OGAWA, TOKYO (JP); HIROTAKA SUZUKI, TOKYO (JP); NORIKO TOTSUKA, TOKYO (JP); TOBIAS CINCAREK, TOKYO (JP); KANAKO WATANABE, TOKYO (JP); SHOHEI YAMAMOTO, TOKYO (JP)

(21) Appl. No.: 17/281,580

(22) PCT Filed: Sep. 2, 2019
(86) PCT No.: PCT/JP2019/034354

§ 371 Date: Mar. 31, 2021

(30) Foreign Application Priority Data
Oct. 12, 2018 (JP) 2018-193428

(51) Int. Cl. G16H 20/60; A47J 36/32; B25J 11/00; G16H 10/60; G06Q 30/06; G06Q 50/12; G05B 19/042

(52) U.S. Cl. CPC G16H 20/60; A47J 36/32; B25J 11/0045; G16H 10/60; G05B 2219/2643; G06Q 30/0633; G06Q 50/12; G05B 19/042; G06Q 30/0631

(57) ABSTRACT

Provided is an information processing apparatus including a control unit (200) that determines a recipe on a basis of user information including an avoidance level of a user for a predetermined ingredient, food information regarding each food, and ingredient information including information regarding each ingredient, in which the avoidance level for the predetermined ingredient is set on a basis of an avoidance factor of the user for the predetermined ingredient, and the food information includes information indicating the ingredient used to cook the food.

### FIELD

The present disclosure relates to an information processing apparatus, an information processing method, and a program.

### BACKGROUND

In recent years, technologies that can automatically change cooking recipes have been developed. For example, Patent Literature 1 discloses a technology for changing a cooking recipe on the basis of conditions such as a user-specified amount and calorie restriction.

### CITATION LIST

### Patent Literature

Patent Literature 1: JP 2003-50907 A

### SUMMARY

### Technical Problem

By the way, the degree of avoidance differs depending on a factor that causes a user to avoid an ingredient. However, in a case of the technology described in Patent Literature 1, since cooking recipes are changed statically, it is not always beneficial to the user.

### Solution to Problem

For solving the problem described above, an information processing apparatus according to one aspect of the present disclosure has a control unit that determines a recipe on a basis of user information including an avoidance level of a user for a predetermined ingredient, food information regarding each food, and ingredient information including information regarding each ingredient, wherein the avoidance level for the predetermined ingredient is set on a basis of an avoidance factor of the user for the predetermined ingredient, and the food information includes information indicating the ingredient used to cook the food.

An information processing method according to one aspect of the present disclosure comprises, determining, by a processor, a recipe on a basis of user information including an avoidance level of a user for a predetermined ingredient, food information regarding cooking of each food, and ingredient information including information regarding each ingredient, wherein the avoidance level for the predetermined ingredient is set on a basis of an avoidance factor of the user for the predetermined ingredient, and the food information includes information indicating the ingredient used to cook the food.

A program according to one aspect of the present disclosure causes a computer to function as an information processing apparatus includes a control unit that determines a recipe on a basis of user information including an avoidance level of a user for a predetermined ingredient, food information regarding cooking of each food, and ingredient information including information regarding each ingredient, wherein the avoidance level for the predetermined ingredient is set on a basis of an avoidance factor of the user for the predetermined ingredient, and the food information includes information indicating the ingredient used to cook the food.

### BRIEF DESCRIPTION OF DRAWINGS

FIG. 1 is a diagram for describing an overview according to an embodiment of the present disclosure.

FIG. 2 is a diagram for describing a functional configuration example of a cooking system according to the present embodiment.

FIG. 3 is a diagram for describing an example of a food information DB 410 in the cooking system according to the embodiment.

FIG. 4 is a diagram for describing an output of a screen of a food order using a food name according to the embodiment, the output being performed by an output unit 600 under the control of a control unit 200.

FIG. 5 is a diagram for describing an output of a screen of a food order including an order condition according to the embodiment, the output being performed by the output unit 600 under the control of the control unit 200.

FIG. 6 is a diagram for describing an example of an ingredient information DB 420 in the cooking system according to the embodiment.

FIG. 7 is a diagram for describing an example of a user information DB 430 in the cooking system according to the embodiment.

FIG. 8 is a diagram for describing an example of registration of a “favorite ingredient”, a “least favorite ingredient”, and an “ingredient to which the user has an allergy” as user information according to the embodiment.

FIG. 9 is a diagram for describing an example of an avoidance level according to the embodiment.

FIG. 10 is a diagram for describing a user information update based on a feedback on a predetermined food from the user according to the embodiment.

FIG. 11 is a diagram for describing a user information update based on a feedback on a predetermined ingredient from a user according to the embodiment.

FIG. 12 is a diagram for describing an example of estimating user information regarding a user from user information of another user according to the embodiment.

FIG. 13 is a diagram for describing an example of a flow of an operation related to recipe determination performed by the control unit 200 with an emphasis on a nutritional balance according to the embodiment.

FIG. 14 is a diagram for describing an example of a flow of an operation related to recipe determination performed by the control unit 200 with an emphasis on a preference according to the embodiment.

FIG. 15 is a diagram for describing an operation related to determination of a recipe further based on the number of food orders and provision of a food based on a response to a recipe recommendation by the cooking system according to the embodiment.

FIG. 16 is a diagram for describing a recipe recommendation by the control unit 200 in a case where there is no order from a user for a predetermined time according to the embodiment.

FIG. 17 is a diagram for describing a flow of an operation related to a case where there is no order from a user for a predetermined time according to the embodiment, the operation being performed by the control unit 200.

FIG. 18 is a diagram for describing an example of notification related to ingredient substitution according to the embodiment, the notification being performed by the control unit 200.

FIG. 19 is a diagram for describing a display control related to user information according to the embodiment, the display control being performed by the control unit 200.

FIG. 20 is an example for describing an example of recipe determination performed by the control unit 200 in a case where a user to be provided with a food has an accompanying person according to the embodiment.

FIG. 21 is a block diagram illustrating a hardware configuration example of an information processing apparatus including the control unit 200 according to an embodiment of the present disclosure.

### DESCRIPTION OF EMBODIMENTS

Hereinafter, a preferred embodiment of the present disclosure will be described in detail with reference to the appended drawings. Note that, in the present specification and the drawings, components having substantially the same functional configuration are provided with the same reference signs, so that repeated description of these components is omitted.

Note that the description will be provided in the following order.

1. Embodiment

1.1. Overview

1.2. Functional Configuration Example

1.3. Specific Example

2. Hardware Configuration Example

3. Conclusion

### 1. EMBODIMENT

### 1.1. Overview

First, the outline of the present disclosure will be described. In recent years, a technology for automatically determining a cooking recipe on the basis of a user-specified condition has been developed. Here, the user-specified condition includes information regarding user's likes and dislikes, allergies, calorie restriction, and the like. In a case of the technology described in Patent Literature 1, the cooking recipe can be changed on the basis of the number of people specified by a user and an order condition such as calorie restriction. Here, an item changed in the cooking recipe is, for example, the kind or amount of an ingredient used in a food.

By the way, in determining a cooking recipe, the degree of avoidance of a predetermined ingredient varies depending on an avoidance factor. However, in the technology described in Patent Literature 1, since the change of the item in the cooking recipe is statically determined, the determined cooking recipe is not always beneficial to the user. For example, in a situation where foods are continuously provided such as a course meal, in a case where all the foods provided are changed to use ingredients having the taste preferred by the user, the satisfaction of the user is not necessarily high.

Further, in addition to determining only a recipe with a taste preferred by the user, nutrients contained in an ingredient can be beneficial to the user. Even in a case of an ingredient avoided by the user, it is desirable to change handling thereof depending on an avoidance factor. For example, in a case of an ingredient to which the user has an allergy, the degree of avoidance thereof in determining a recipe is high. However, for example, in a case of an ingredient that the user cannot eat in its original form, as the ingredient is processed so that the ingredient does not remain in its original form, rather than determining a recipe so as to avoid the ingredient, a possibility that the user can eat the ingredient increases. In a case where the ingredient contains nutrients that the user needs, it is beneficial to the user.

Therefore, in the present disclosure, a recipe suitable for each user can be determined by using user information including an avoidance level of a user for a predetermined ingredient and considering a different type of order condition from an order condition included in a food order. Further, by using an avoidance level that is set on the basis of an avoidance factor of a user for a predetermined ingredient, it is possible to determine a recipe suitable for the user even in a case where order conditions that are difficult to compare are mixed.

Here, the avoidance level refers to the degree to which a predetermined ingredient is not desired to be included in a food. Here, the avoidance factor refers to a factor of avoidance of a predetermined ingredient. Examples of the avoidance factor include a dislike of taste, an allergy, and a dislike of texture. Details of the avoidance level and avoidance factor will be described later.

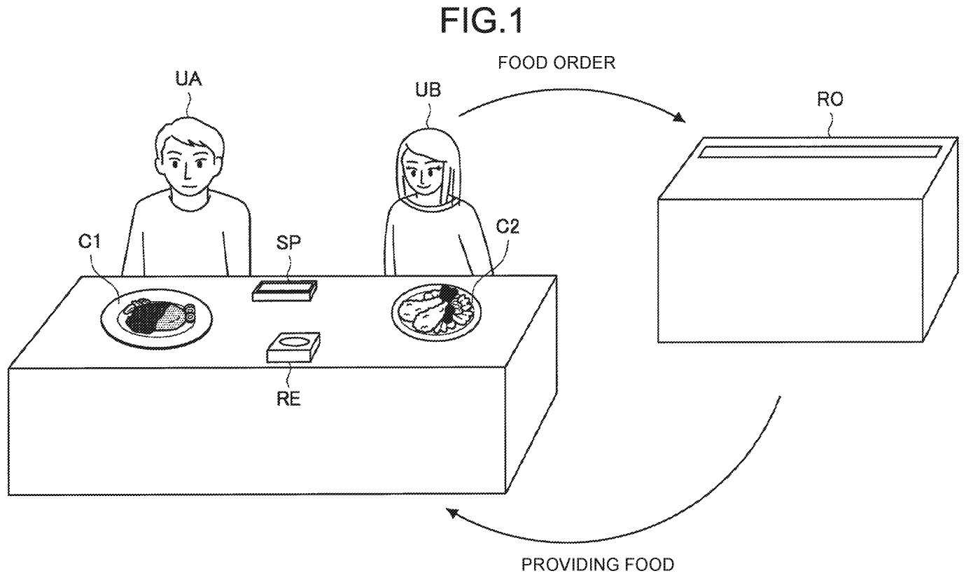

Next, an overview according to an embodiment of the present disclosure will be described. A cooking system according to an embodiment of the present disclosure is assumed to be used in a facility such as a restaurant or a home having a cooking robot. FIG. 1 is a diagram for describing an overview according to an embodiment of the present disclosure. FIG. 1 illustrates a user UA, a user UB, and a cooking robot RO.

First, the users UA and UB register their user information regarding a food or ingredient in an information processing apparatus (not illustrated in FIG. 1). For example, the users UA and UB may register the user information by performing an input operation on a predetermined application running on a smartphone SP illustrated in FIG. 1. Here, a reader RE is a device capable of reading user information from the smartphone SP and transmitting the user information to a cooking server, for example, by using non-contact wireless communication.

Next, the users UA and UB place a food order. The users UA and UB can place a food order by using the smartphone SP. For example, the users UA and UB may place a food order by performing an input operation on a predetermined application running on the smartphone SP. Further, the users UA and UB may place a food order by putting the smartphone SP on the reader RE illustrated in FIG. 1, similarly to the registration of the user information.

The information processing apparatus (hereinafter, also referred to as the cooking server) determines a recipe on the basis of the acquired food order. Once the users UA and UB place a food order, the cooking robot RO illustrated in FIG. 1 performs cooking by using the recipe determined on the basis of the food order. The cooked food is provided to the user by a person or by a robot.

Note that, in the example of FIG. 1, the user UA ordered a “hamburger steak” as an order condition. Here, the cooking server determines a recipe for a cheese-free hamburger steak C1 on the basis of information indicating that the user UA has a cheese allergy, and the cooking robot performs cooking on the basis of the recipe and provides the cooked food. Further, the user UB ordered a “shrimp” as an order condition. Here, the cooking server determines a recipe for a cooked shrimp C2 on the basis of preset information indicating that the user UB dislikes a “raw” ingredient, and the cooking robot performs cooking on the basis of the recipe and provides the cooked food.

Note that the food order transmitted from the smartphone SP may be received by the cooking server. Further, the users UA and UB may place a food order by voice using a microphone included in the smartphone SP.

Here, the user information includes information indicating an avoidance factor and an avoidance level of the user for a predetermined ingredient. The cooking server can determine a recipe suitable for each user by using the user information including the information indicating the avoidance factor and the avoidance level. Details of the user information will be described later.

Hereinabove, the overview of the cooking system according to an embodiment of the present disclosure has been described.

<<1.2. Functional Configuration Example>>

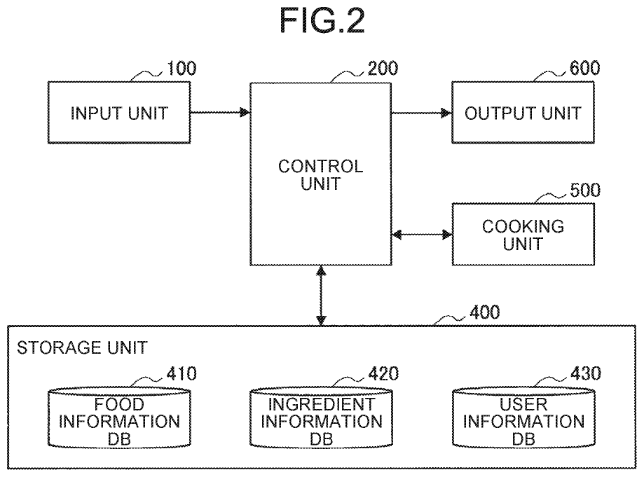

Next, a functional configuration example of the cooking system according to the present embodiment will be described. FIG. 2 is a diagram for describing the functional configuration example of the cooking system according to the present embodiment. The cooking system includes an input unit 100, a control unit 200, a storage unit 400, a cooking unit 500, and an output unit 600. Note that, hereinafter, the food order is not limited to an order that specifies a food name, but refers to an order that includes conditional orders such as information regarding a user preference and an allergy, and calorie restriction.

The input unit 100 according to the present embodiment has a function of receiving a food order from a user.

The input unit 100 may be included in the smartphone SP illustrated in FIG. 1 as described above, and may receive an input operation from the user, the input operation being performed on the predetermined application run by the smartphone SP. Further, the input unit 100 may receive a food order placed by voice using the microphone included in the smartphone SP as described above.

The input unit 100 may receive, as the food order, a food name specified by the user, and may receive, for example, information regarding an order condition of the user.

The control unit 200 according to the present embodiment has a function of determining a recipe on the basis of the food order acquired from the input unit 100. Specifically, the control unit 200 analyzes a content of the food order acquired from the input unit 100 and determines a content input by the user. Further, the control unit 200 may determine a recipe on the basis of other information in addition to the food order. The other information is stored in, for example, the storage unit 400 as described later. The control unit 200 appropriately acquires information from the storage unit 400 and determines a recipe. Further, the control unit 200 can update data stored in the storage unit 400. Note that the control unit 200 is included in the information processing apparatus that is independent of the input unit 100, the cooking unit 500, and the output unit 600.

The storage unit 400 according to the present embodiment has a function of storing various information used by the control unit 200 to determine a recipe and providing various information to the control unit 200 under the control of the control unit 200. The storage unit 400 includes a food information DB 410, an ingredient information DB 420, and a user information DB 430. In addition, the storage unit 400 stores an order history of each user. The order history is a history of food orders placed by each user.

Here, the food information DB 410 stores food information data regarding each food. The food information includes a recipe corresponding to each food. The ingredient information DB 420 stores ingredient information data including information regarding each ingredient. The information regarding each ingredient includes, for example, information regarding a nutritional value of each ingredient. The user information DB 430 stores user information data including information regarding a predetermined ingredient of the user. Details of each of the above-described databases (DBs) will be described later. Note that the storage unit 400 may be included in the information processing apparatus including the control unit 200.

The cooking unit 500 according to the present embodiment is an automatic cooking device having a function of performing cooking on the basis of the recipe determined by the control unit 200. The cooking unit 500 may be, for example, a cooking robot and be independent of other components.

The output unit 600 according to the present embodiment has a function of outputting predetermined information to the user under the control of the control unit 200. Specifically, the output unit 600 outputs, to the user, information regarding the recipe determination performed by the control unit 200, for example, food information stored in the food information DB 410.

The output unit 600 may be included in the smartphone SP illustrated in FIG. 1 as described above, and may display the predetermined information on a screen of the predetermined application run by the smartphone SP. Further, as described above, the input unit 100 may output the predetermined information to the user by using, for example, a speaker included in the smartphone SP.

Hereinabove, the configuration of the cooking system according to the present embodiment has been described. Note that the configuration described above with reference to FIG. 2 is merely an example, and the functional configuration of the cooking system according to the present embodiment is not limited to the example.

<<1.3. Specific Example>>

Subsequently, the databases (DBs) included in the storage unit 400 according to the present embodiment will be described in detail. In addition, the use of data in the recipe determination performed by the control unit 200 according to the present embodiment will be described in detail. Moreover, the output of information regarding the data according to the present embodiment will described in detail, the information being output by the output unit 600.

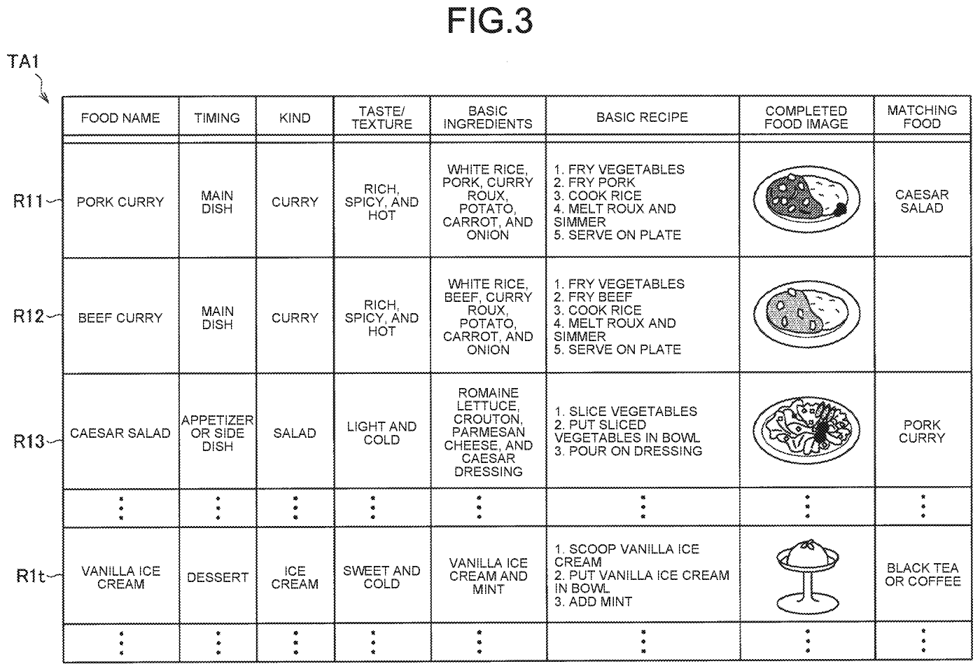

FIG. 3 is a diagram for describing an example of the food information DB 410 in the cooking system according to the present embodiment. FIG. 3 illustrates a food information table TA1 stored in the food information DB 410. The food information table TA1 shows food information records R11 to R13 and R1t as an example.

The food information table TA1 includes information regarding each food. The information regarding each food is stored in each of the food information records R11 to R13, R1t, and the like. The control unit 200 determines a recipe by referring to the food information table TA1. The control unit 200 may refer to each attribute (column) as necessary when determining the recipe.

Hereinafter, each attribute (column) in the example illustrated in FIG. 3 will be described.

A “food name” column shows food names. For example, a “food name” in the food information record R11 is “pork curry”. Note that, in the “food name” column, the names of foods that can be provided are shown, and the names of foods that cannot be provided may also be shown.

A “timing” column shows at which timing each food needs to be provided, for example, in a case of a course meal. The control unit 200 may determine a recipe by referring to data in the “timing” column. For example, a “timing” in the food information record R11 is “main dish”, and a “timing” in the food information record R13 is “appetizer or side dish”. Note that the “timing” may be the number of ordered items, a time elapsed since the first order was placed, or the like.

A “type” column shows the kinds into which respective foods are roughly classified, and a “taste/texture” column shows the taste and texture. For example, a “kind” in the food information record R11 is “curry”, and a “taste/texture” is “rich, spicy, and hot”. The “kind” column and the “taste/texture” column are used when determining a recipe for a food order including user-specified order conditions such as the kind, taste, and texture.

A “basic ingredients” column shows an ingredient included in each food. When performing cooking, the cooking unit 500 uses an ingredient by referring to a “basic ingredients” column in a food information record of a food to be cooked. The kind of the ingredient shown in the “basic ingredients” column corresponds to the kind of the ingredient in ingredient information stored in the ingredient information DB 420. For example, “basic ingredients” in the food information record R11 are “white rice, pork, curry roux, potato, carrot, and onion”.

A “basic recipe” column shows a cooking method of each food. When performing cooking, the cooking unit 500 performs cooking by referring to a “basic recipe” column in a food information record of a food to be cooked. For example, a “basic recipe” in the food information record R11 is “1. fry vegetables; 2. fry pork; 3. cook rice; melt roux and simmer; and 5. serve on a plate”. Note that, in the “basic recipe” column, a cooking method of each food may be stored in more detail than that in the example illustrated in FIG. 3.

In a “completed food image” column, data that visually shows an example of a completed form of each food is stored. Here, the example of the completed form stored in the “completed food image” column may be, for example, photographic data. In addition, the example of the completed form may be one in which each ingredient is arranged by using computer graphics (CG). For example, in a case where the control unit 200 determines a recipe in which a predetermined ingredient is substituted, the output unit 600 may replace a CG image of the ingredient stored in the ingredient information DB 420 with a CG image of a substitute ingredient and output a completed food image to the user.

A “matching food” column shows the name of a food that matches with each food. Here, the “matching” means that, for example, foods do not cause a problem when provided at the same time, or a food is suitable to be provided to take away the aftertaste after a predetermined food is provided. For example, a “matching food” in the food information record R11 is “Caesar salad”, and a “matching food” in the food information record R13 is “pork curry”. The food name stored in the “matching food” column is not limited to the above example.

Hereinabove, an example of the food information table TA1 stored in the food information DB 410 has been described. Note that the storage unit 400 may store the food information table TA1 stored in the food information DB 410 in a format that can be searched for and analyzed by the control unit 200, in addition to a table format as illustrated in FIG. 3. For example, the storage unit 400 may store the food information table TA1 in the food information DB 410 in a form of YAML Ain′t Markup Language (YAML).

Note that the food information DB 410 may be prepared in advance or may be added sequentially. Further, the above-described attribute example is only an example, and is not limited to such an example.

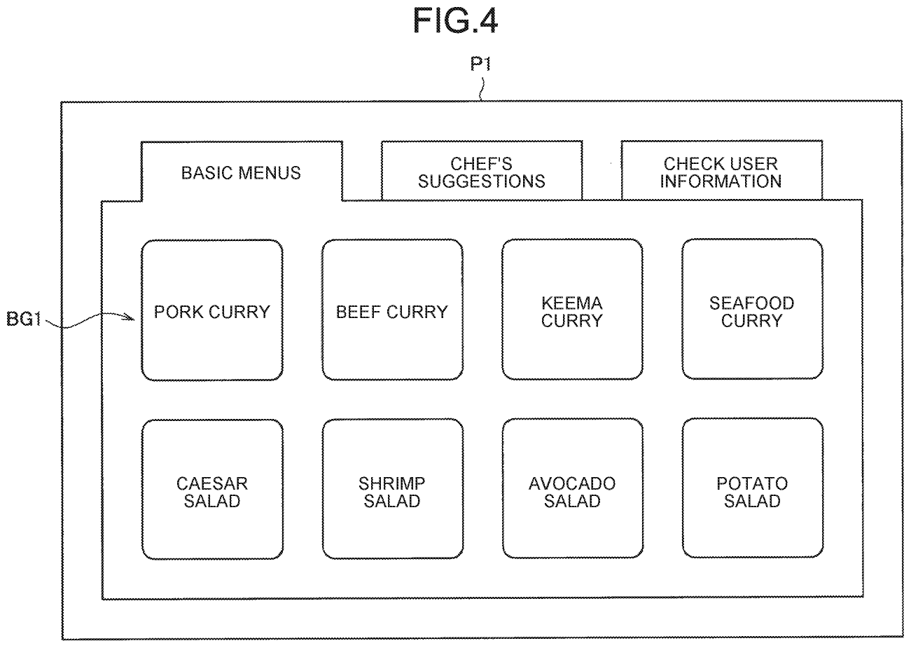

Next, an output of a screen of a food order using a food name will be described, the output being performed by the output unit 600 under the control of the control unit 200. FIG. 4 is a diagram for describing the output of a screen of a food order using a food name according to the embodiment, the output being performed by the output unit 600 under the control of the control unit 200. FIG. 4 illustrates an output screen P1. An order button group BG1 is shown in the output screen P1. Note that the output screen P1 may be output by, for example, the smartphone SP illustrated in FIG. 1.

Here, the order button group BG1 means a set of a plurality of order buttons. When the user touches one of the plurality of order buttons, a food corresponding to the touched order button is ordered. In FIG. 4, when the user touches any of the order buttons, a food with a food name written on the touched order button is ordered.

In the example of FIG. 4, when the user touches a button on which pork curry is written, the input unit 100 acquires a food order of “pork curry”. The control unit 200 determines a recipe on the basis of the food order of “pork curry” acquired by the input unit 100. Note that, in addition to the food name, the food order may include information indicating which user placed the food order.

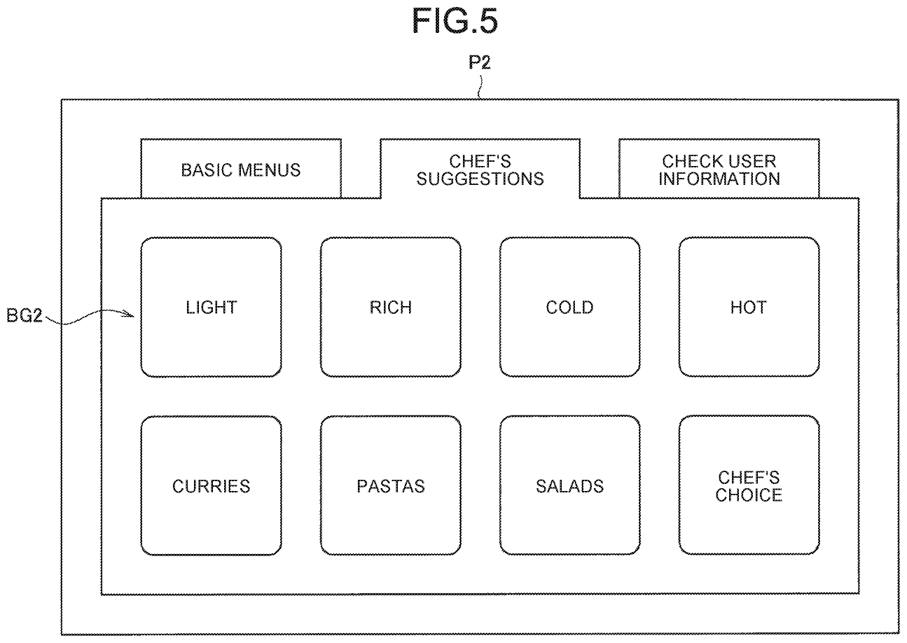

Hereinabove, the output of a screen of a food order using a food name has been described. Next, an output of a screen of a food order including an order condition will be described, the output being performed by the output unit 600 under the control of the control unit 200. FIG. 5 is a diagram for describing the output of a screen of a food order including an order condition according to the present embodiment, the output being performed by the output unit 600 under the control of the control unit 200. FIG. 5 illustrates an output screen P2. An order button group BG2 is shown in the output screen P2.

In the example of FIG. 5, when an order button on which “light foods” is written is touched, the input unit 100 acquires a food order including an order condition of “light foods”. The control unit 200 determines a recipe on the basis of the food order. At this time, the control unit 200 is illustrated in FIG. 3. The control unit 200 determines a recipe of which the “taste/texture” column is “light foods” in the food information table TA1. Note that the control unit 200 may determine a recipe on the basis of a condition other than the condition that the “taste/texture” column is “light foods”.

In this way, even in a case where the user places an ambiguous order, the control unit 200 can determine a suitable recipe by using the food information DB 410.

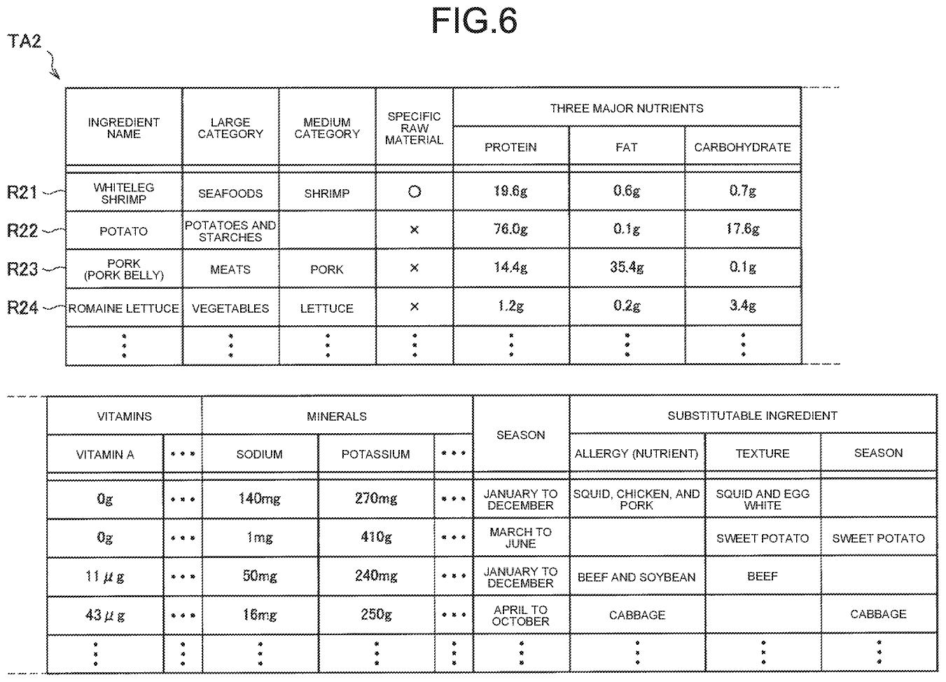

Subsequently, an example of the ingredient information DB 420 in the cooking system according to the present embodiment will be described. FIG. 6 is a diagram for describing an example of the ingredient information DB 420 in the cooking system according to the present embodiment. FIG. 6 illustrates an ingredient information table TA2 stored in the ingredient information DB 420. The ingredient information table TA2 includes food information records R21 to R24 as an example.

The ingredient information table TA2 includes information regarding each food. The information regarding each food is stored in each of the food information records R21 to R24 and the like. The control unit 200 determines a recipe by referring to the ingredient information table TA2. When determining the recipe, the control unit 200 refers to a food information record of the ingredient information table TA2 as necessary, the food information record corresponding to an ingredient name shown in the “basic ingredients” column of the food information table TA1.

Hereinafter, each attribute (column) in the example illustrated in FIG. 6 will be described.

An “ingredient name” column shows the name of each ingredient. For example, an “ingredient name” of the ingredient information record R21 is “whiteleg shrimp”.

A “large category” column and a “medium category” column show the kinds into which respective ingredients are roughly classified. The “medium category” column shows the kinds into which the ingredients are more specifically classified, as compared with those in the “large category” column. For example, a “large category” in the ingredient information record R21 is “seafoods”, and a “medium category” is “shrimp”.

A “specific raw material” column shows whether or not each ingredient is a specific raw material. Here, the specific raw material means an ingredient that causes a food allergy. For example, a “specific raw material” in the ingredient information record R21 is “o (the ingredient is the specific raw material)”.

A “three major nutrients” column, a “vitamins” column, and a “minerals” column show nutritional information of each ingredient. Note that the amount of nutrient shown in the example of FIG. 6 is the amount of nutrient per 100 g. The control unit 200 may determine a recipe by referring to the “three major nutrients” column, the “vitamins” column, and the “minerals” column of the ingredient information table TA2. For example, the control unit 200 may determine a recipe so that a nutrient intake amount of the user reaches an ideal nutrient amount by referring to the “three major nutrients” column, the “vitamins” column, and the “minerals” column of the ingredient information table TA2. Note that information on the amount of nutrient can be based on, for example, publicly known nutritional value information.

A “season” column shows the season of each ingredient. The control unit 200 may determine a recipe so that a food including a seasonal ingredient can be provided to the user on the basis of the “season” column of the ingredient information DB 420. Note that data for an ingredient that is in season all year round may be empty in the “season” column.

A “substitutable ingredient” column shows an ingredient that can substitute for each ingredient. The control unit 200 refers to the “substitutable ingredient” column when determining a recipe in which a predetermined ingredient is substituted. In the “substitutable ingredient” column, ingredients are classified according to a factor of substitution. For example, ingredients are classified into “allergy (nutrient)”, “texture”, and “season”. For example, “allergy (nutrient)” shows a substitutable ingredient in a case where a corresponding ingredient is an ingredient to which the user has an allergy. In the ingredient information table TA2, items for which substitute ingredients are not registered are blank. Further, for example, a “texture” of a “substitutable ingredient” of the ingredient information record R21 is “squid and egg white”.

Note that the number of classifications in the “substitutable ingredient” column may be adjusted to be less than or equal to a predetermined number in order to avoid frequent substitution of ingredients. In addition, the classification in the “substitutable ingredient” column may be used for purposes other than those described in the classification. For example, the ingredient shown in “allergy (nutrient)” may substitute for an ingredient “that the user can eat if the taste is covered up”.

Note that the ingredient information DB 420 may be prepared in advance or may be added sequentially. Further, the above-described attribute example is only an example, and is not limited to such an example. Another example of the attribute includes “possibility of being eaten raw”. In a case of fishes or the like, even when the “kinds” of all ingredients are “fishes”, some ingredients may be more suitable to be eaten raw and some ingredients may be more suitable to be eaten by heating. Therefore, proper cooking can be performed by referring to “possibility of being eaten raw”.

Further, the ingredient information DB 420 may include inventory information of each ingredient. The control unit 200 can determine a recipe of a food that can be currently provided by referring to the inventory information of each ingredient. In addition, the ingredient information DB 420 may include the freshness of a predetermined ingredient such as vegetables. The control unit 200 can perform inventory management and provide a fresher ingredient by referring to the freshness of the predetermined ingredient. In addition, the ingredient information DB 420 may include a CG image of each ingredient. The CG image of each ingredient may be used in the “completed food image” of the food information DB 410.

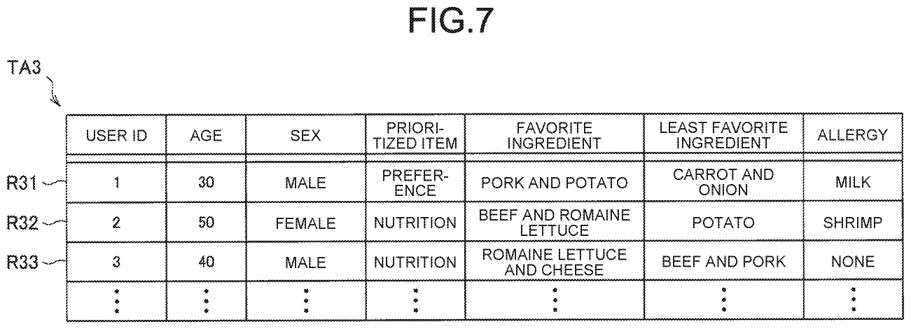

Subsequently, an example of the user information DB 430 in the cooking system according to the present embodiment will be described. FIG. 7 is a diagram for describing an example of the user information DB 430 in the cooking system according to the present embodiment. FIG. 7 illustrates a user information table TA3 stored in the user information DB 430. The user information table TA3 includes user information records R31 to R33 as an example.

The user information table TA3 includes information regarding each food. The information regarding each food is stored in each of the user information records R31 to R33 and the like. The control unit 200 determines a recipe by referring to the user information table TA3. When determining the recipe, the control unit 200 refers to a user information record of the user information table TA3 as necessary, the user information record corresponding to a target user for which the recipe is determined in the user information table TA3.

Hereinafter, each attribute (column) in the example illustrated in FIG. 6 will be described. A “user ID” column shows a number or character string assigned to each user. The control unit 200 can uniquely specify the user by referring to the “user ID” column.

An “age” column and a “sex” column show additional information of each user. The control unit 200 may determine a recipe on the basis of the additional information. On the basis of a plurality of pieces of additional information, the control unit 200 may estimate data of a “favorite ingredient” column of a predetermined user from a user information record of another user similar to the predetermined user, the “favorite ingredient” column being to be described later. For example, an “age” in the user information record R31 is “30” and a “sex” is “male”.

A “prioritized item” column shows a tendency of the priority in each user's diet. Examples of the prioritized item include preference and nutrition. Here, the preference as the prioritized item indicates that the user has a tendency to eat by giving priority to the preference of the user in the taste or texture, rather than a nutritional balance. On the other hand, here, the nutrition as the prioritized item indicates that the user has a tendency to eat in consideration of a nutritional balance, rather than the preference of the user in the taste or texture. When determining a recipe, the control unit 200 may refer to the “prioritized item” column and determine the recipe according to the tendency of each user. For example, a “prioritized item” in the user information record R31 is “preference”. Note that the tendency shown in the “prioritized item” column is not limited to the above example.

In the example of FIG. 7, the “prioritized item” column in the user information record R31 is “preference”, while a “prioritized item” column in the user information records R31 and R32 is “nutrition”.

A “favorite ingredient” column and a “least favorite ingredient” column show preferences of the user for a predetermined ingredient, each of which is one of the avoidance factors. The “favorite ingredient” column shows the name of an ingredient preferred by the user. On the other hand, the “least favorite ingredient” column shows the name of an ingredient avoided by the user. When determining a recipe, the control unit 200 refers to the “favorite ingredient” column and the “least favorite ingredient” column, and may determine, for example, a recipe that includes an ingredient preferred by the user and does not include an ingredient avoided by the user. For example, a “favorite ingredient” in the user information record R31 is “pork and potato”, and a “least favorite ingredient” is “carrot and onion”.

Note that, in the example of FIG. 7, the “favorite ingredient” column and the “least favorite ingredient” column are each shown as a single column data, but ingredients may be classified more specifically for each avoidance factor. For example, in the “favorite ingredient” column, ingredients may be classified into “taste”, “texture”, and the like which are factors that make the user like the ingredients.

An “allergy” column shows the kind of an ingredient to which each user has an allergy as the avoidance factor. When determining a recipe, the control unit 200 may refer to the “allergy” column and determine a recipe that includes only an ingredient to which the user does not have an allergy. For example, an “allergy” in the user information record R31 is “milk”.

Further, although not illustrated in FIG. 7, examples of the information included in the user information table TA3 may include credit card information, an access right to a schedule, and pre-checking of foods.

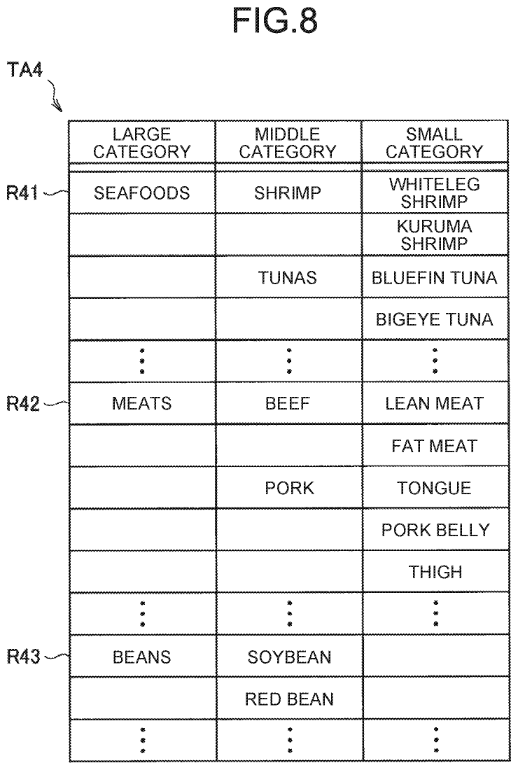

Here, registration of a “favorite ingredient”, a “least favorite ingredient”, and an “ingredient to which the user has an allergy” as the user information will be described. FIG. 8 is a diagram for describing an example of the registration of a “favorite ingredient”, a “least favorite ingredient”, and an “ingredient to which the user has an allergy” as the user information according to the present embodiment. FIG. 8 illustrates an ingredient classification table TA4 in which ingredients are classified into categories. The ingredient classification table TA4 shows large category information records R41 to R43 as an example. FIG. 8 illustrates a “large category” column in which ingredients are roughly classified, a “medium category” column in which the kinds of the ingredients shown in the large category are more specifically classified, and a “small category” column in which the kinds of the ingredients shown in the medium category are more specifically classified.

The input unit 100 may receive registration of a “favorite ingredient”, a “least favorite ingredient”, and an “ingredient to which the user has an allergy” through an input operation performed by the user on, for example, the predetermined application running on the smartphone SP illustrated in FIG. 1. At this time, the user may register an ingredient in any one of the large category, medium category, and small category. In addition to the user, the ingredient information table TA2 may register a category of each ingredient on the basis of the category classification in the ingredient classification table TA4.

In the example of FIG. 8, a “large category” of the large category information record R41 is “seafoods”. A “medium category”, which is the “large category” and corresponds to “seafoods”, is “shrimp and tunas”. A “small category” corresponding to “shrimp” which is the “medium category” is “whiteleg shrimp and kuruma shrimp”.

Next, information regarding a predetermined ingredient included in the user information table TA3 will be described. In FIG. 7, each user has registered a predetermined ingredient as one of a “favorite ingredient”, a “least favorite ingredient”, or an “ingredient to which the user has an allergy”. Here, the predetermined ingredient is set as information having a plurality of levels.

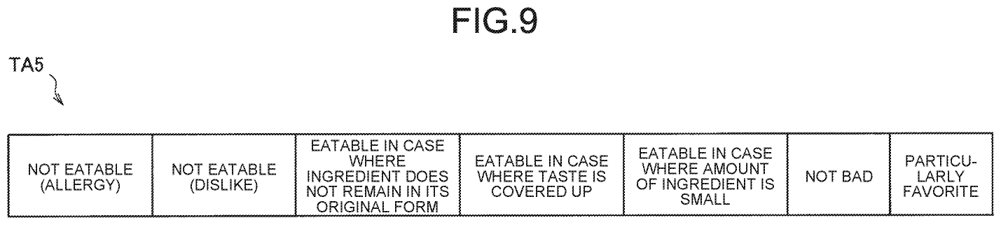

FIG. 9 is a diagram for describing an example of the avoidance level according to the present embodiment. FIG. 9 illustrates an example of a plurality of levels that constitute avoidance levels. The control unit 200 acquires a corresponding avoidance level on the basis of an avoidance factor of each user for a predetermined ingredient, the avoidance factor being acquired by the input unit 100. That is, in a case where avoidance factors of each user for a predetermined ingredient are different, the control unit 200 acquires different avoidance levels.

An example of FIG. 9 will be described. FIG. 9 illustrates a table TA5 showing a plurality of levels that constitute avoidance levels. “Not eatable (allergy)”, which is an avoidance factor having a level shown on the leftmost side of FIG. 9, corresponds to the highest avoidance level, and a level on the right side corresponds to a lower avoidance level. “Particularly favorite”, which is an avoidance factor having a level shown on the rightmost side of FIG. 9, corresponds to the lowest avoidance level.

For example, in FIG. 9, the control unit 200 can compare an ingredient that is “eatable in a case where the ingredient does not remain in its original form” and an ingredient that is “eatable in a case where the taste is covered up” with each other. In the table TA5, an avoidance level set on the basis of the avoidance factor “eatable in a case where the ingredient does not remain in its original form” is higher than an avoidance level set on the basis of the avoidance factor “eatable in a case where the taste is covered up”. That is, the control unit 200 can preferentially avoid, on the basis of the table TA5, an ingredient that is “eatable in a case where the ingredient does not remain in its original form”, rather than an ingredient that is “eatable in a case where the taste is covered up”.

Note that the control unit 200 may perform ingredient substitution so that an avoidance level for an ingredient is lower than or equal to a predetermined level. For example, in the example of FIG. 9, the control unit 200 may actively perform ingredient substitution in a case where the avoidance level is higher than or equal to the avoidance level corresponding to “eatable in a case where the taste is covered up”. On the other hand, the control unit 200 does not have to perform ingredient substitution in a case where the ingredient is used in such a manner as to eliminate the avoidance factor in a predetermined recipe. For example, in a recipe in which an ingredient that is “eatable in a case where the ingredient does not remain in its original form” is used in such a manner that the ingredient does not remain in its original form, the ingredient does not have to be substituted. Further, in a case where ingredient substitution occurs in determining a recipe, the control unit 200 may substitute the ingredient with an ingredient that is considered by the user as being “not bad” or “particularly favorite” in order to further lower the avoidance level of the user. Note that the avoidance level may be stored in the user information DB 430, or may be set on the basis of the avoidance factor when the control unit 200 determines a recipe. Further, the above-described determination performed by the control unit 200 is not limited to such an example.

As such, it is possible to determine a recipe that is more suitable for each user by setting the avoidance level on the basis of the avoidance factor. With such a function, it becomes possible to determine a recipe that is more beneficial to the user.

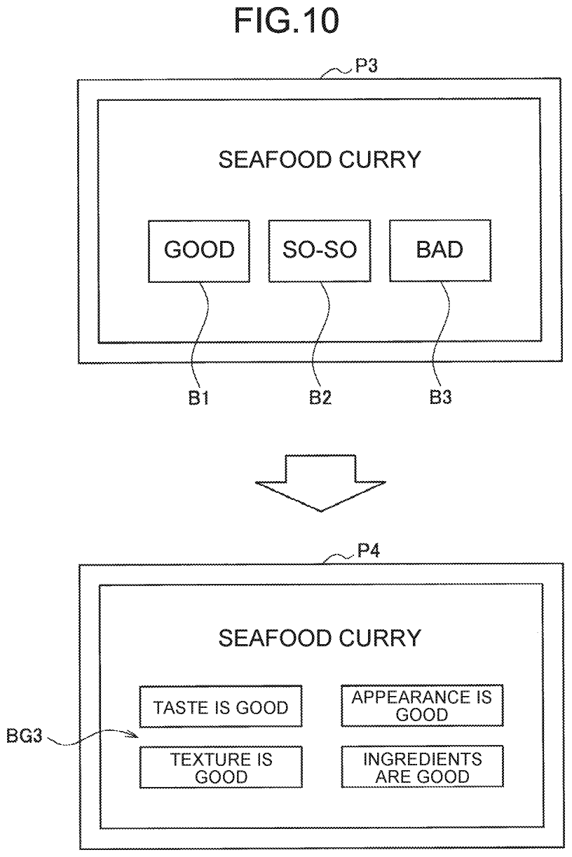

Subsequently, a user information update based on a feedback from the user will be described, the user information update being performed by the control unit 200. The control unit 200 according to the present embodiment may update user information of a user on the basis of a feedback from the user. FIG. 10 is a diagram for describing a user information update based on a feedback on a predetermined food from the user according to the present embodiment. FIG. 10 illustrates display screens P3 and P4 related to feedbacks. In FIG. 10, the user gives a feedback on a predetermined food by touching any of buttons shown in the display screens P3 and P4.

In the example illustrated on the upper side of FIG. 10, the control unit 200 determines an evaluation on a seafood curry on the basis of a touch on any of buttons B1 to B3, the touch being acquired by the input unit 100 and performed by the user. Note that the control unit 200 may allow the user to perform a more detailed evaluation on a predetermined food. In the example illustrated on the lower side of FIG. 10, the control unit 200 causes the output unit 600 to output a button group BG3. Here, when the user touches a button on which “taste is good” is written, the control unit 200 updates user information of the user so that the user likes the taste of seafood curry.

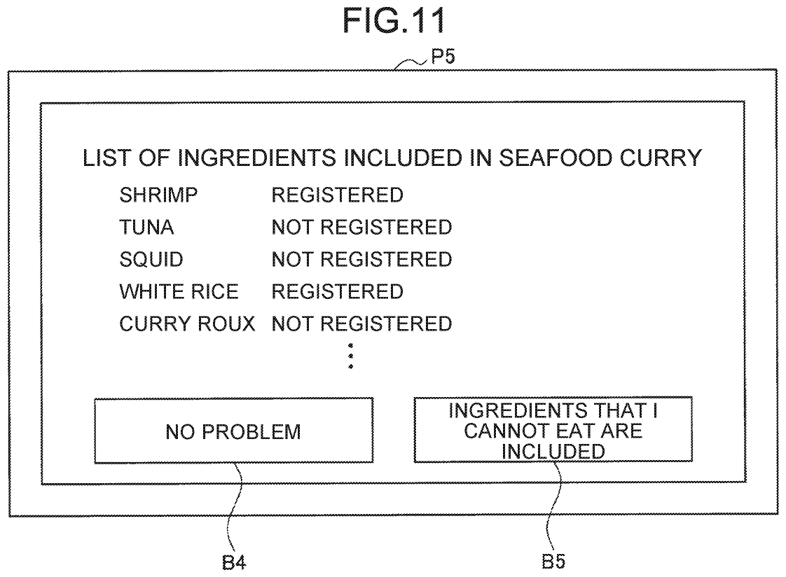

In the above, a feedback on a predetermined food from the user has been described. The control unit 200 may update user information on the basis of a feedback on a predetermined ingredient from the user. FIG. 11 is a diagram for describing a user information update based on a feedback on a predetermined ingredient from the user according to the present embodiment. FIG. 11 illustrates a display screen P5 related to a feedback. In FIG. 11, the user performs an evaluation on a predetermined ingredient by touching any of buttons shown in the display screen P5.

In the example of FIG. 11, the control unit 200 determines an evaluation on ingredients of a seafood curry on the basis of a touch on any of buttons B4 and B5, the touch being acquired by the input unit 100 and performed by the user. Here, when the user touches a button B4 on which “no problem” is written, the control unit 200 updates user information of the user in the user information DB 430 so that all the ingredients of the seafood curry are eatable ingredients. Note that an example of the display performed by the output unit 600 is not limited to such an example. Note that the input unit 100 may receive an utterance of the user as an evaluation on each ingredient included in a predetermined recipe. Note that in a case where the input unit 100 acquires a feedback “problematic” on a substitute ingredient, the control unit 200 may limit the use of a similar ingredient as a substitute in a food that includes (included) the substitute ingredient.

As such, it is possible to obtain more realistic user information and set the avoidance level on the basis of a feedback from the user. With such a function, it becomes possible to determine a recipe that is more suitable for each user.

Further, the control unit 200 may estimate predetermined user information on the basis of user information of another user, which is similar to the predetermined user information.

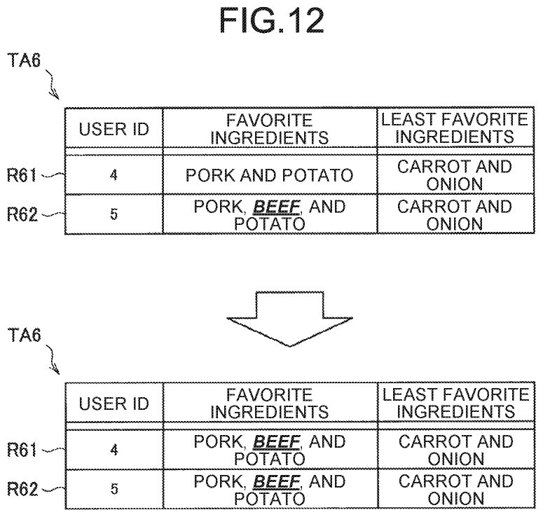

FIG. 12 is a diagram for describing an example of estimating user information regarding a user from user information of another user according to the present embodiment. FIG. 12 illustrates the user information table TA3. Here, the user information table TA3 includes user information records R61 and R62.

An example of FIG. 12 will be described. “Favorite ingredients” and “least favorite ingredients” of user IDs 4 and 5 are the same except for “beef” shown in the “favorite ingredients” of the user ID 5. Here, the control unit 200 may determine that a user with the user ID 4 and a user with the user ID 5 are similar to each other. Next, the control unit 200 may estimate that “beef” shown in the “favorite ingredients” of the user ID 5 is also included in the “favorite ingredients” of the user ID 4. When determining a recipe, the control unit 200 may assume that “beef” is included in the “favorite ingredients” of the user ID 4. In addition, the control unit 200 may cause the output unit 600 to notify the user corresponding to the user ID 4 whether or not to add “beef” to the “favorite ingredients”. When a response from the user to the notification performed by the output unit 600 is received as acceptance, the control unit 200 may assume that “beef” is included in the “favorite ingredient” of the user ID 4.

Note that the control unit 200 may estimate user information by using collaborative filtering.

In this way, it is possible to estimate information regarding a user by using user information of another user, the information having not yet been registered by the user. With such a function, it becomes possible to determine a recipe that is more suitable for the user.

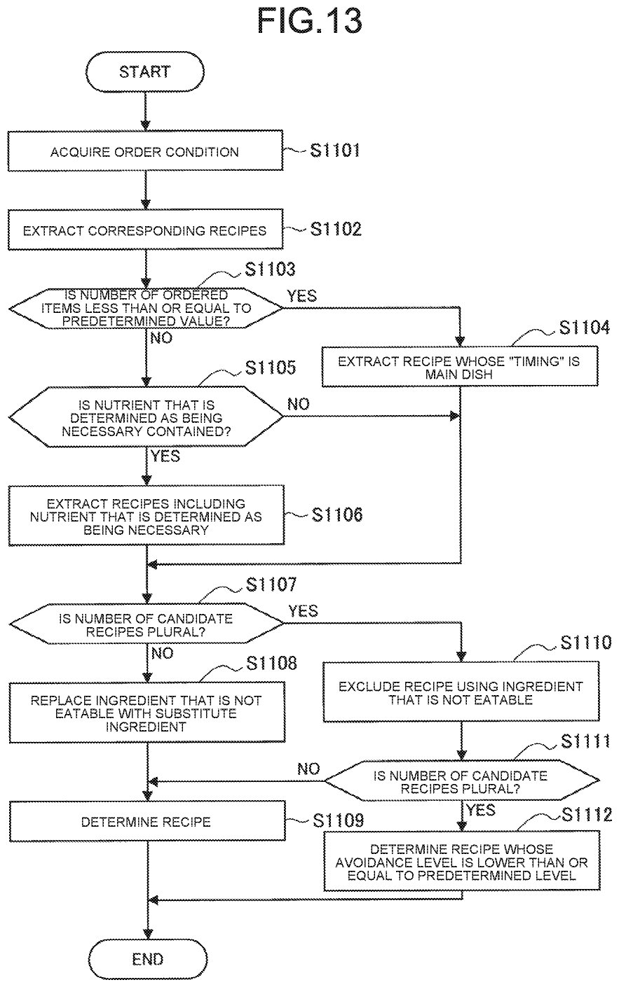

Next, an example of a flow of an operation related to recipe determination performed by the control unit 200 will be described. FIG. 13 is a diagram for describing an example of the flow of the operation related to recipe determination performed by the control unit 200 with an emphasis on a nutritional balance according to the present embodiment.

Referring to FIG. 13, first, the control unit 200 acquires an order condition from the input unit 100 (S1101). Next, the control unit 200 extracts recipes corresponding to the order condition from the food information DB 410 on the basis of the order condition acquired in Step S1101 (S1102). Next, in a case where the number of items ordered by the user is less than or equal to a predetermined value (S1103: YES), the control unit 200 extracts a recipe whose “timing” is a main dish among recipes extracted in Step S1102 (S1104).

On the other hand, in a case where the number of items ordered by the user is not less than or equal to the predetermined value (S1103: NO), the control unit 200 calculates a nutrient intake amount of the user on the basis of the number of items ordered by the user, and determines, on the basis of the calculated intake amount and an ideal nutrient amount of the user, whether or not a necessary nutrient is contained (S1105). In a case where it is determined that the nutrient that is determined as being necessary is not contained (S1105: NO), the control unit 200 determines whether or not the number of candidate recipes is plural (S1107).

On the other hand, in a case where it is determined that the nutrient that is determined as being necessary is contained (S1105: YES), the control unit 200 extracts a recipe containing a predetermined amount or more of nutrient that is determined as being necessary among the candidate recipes (S1106), and determines whether or not the number of extracted recipes is plural (S1107). In a case where it is not determined in Step S1107 that the number of candidate recipes is plural (in a case where the number of candidate recipes is one) (S1107: NO), the control unit 200 replaces an ingredient that is not eatable by the user among ingredients of the candidate recipe with a substitute ingredient (S1108) and determines the recipe (S1109).

On the other hand, in a case where it is determined that the number of candidate recipes is plural (S1107: YES), the control unit 200 excludes a recipe including an ingredient that is not eatable by the user among the candidate recipes (S1110). Next, in a case where it is determined that the number of candidate recipes is not plural (S1111: NO), the control unit 200 determines the recipe (S1108) and ends the operation. On the other hand, in a case where it is determined that the number of candidate recipes is plural (S1111: YES), the control unit 200 determines a recipe so that the avoidance level is lower than or equal to a predetermined level on the basis of another order condition (S1112), and ends the operation.

The above-described flow of the operation related to recipe determination performed by the control unit 200 with an emphasis on a nutritional balance is merely an example, and is not limited to such an example. Further, in a case where there is no candidate recipe in Step S1107, for example, a recipe having the lowest avoidance level may be determined in Step S1102, for example. Further, in Step S1108 or Step S1112, the control unit 200 may notify the user of the determined recipe.

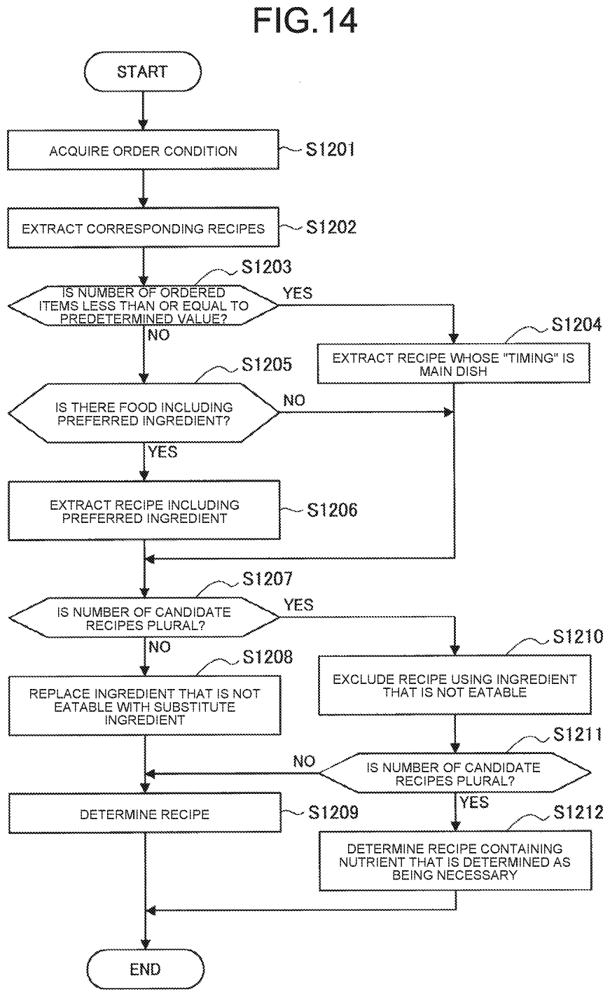

Next, an example of a flow of an operation related to recipe determination performed by the control unit 200 with an emphasis on a preference will be described. FIG. 14 is a diagram for describing an example of the flow of the operation related to recipe determination performed by the control unit 200 with an emphasis on a preference according to the present embodiment.

Referring to FIG. 14, first, the control unit 200 acquires an order condition from the input unit 100 (S1201). Next, the control unit 200 extracts recipes corresponding to the order condition from the food information DB 410 on the basis of the order condition acquired in Step S1101 (S1202). Next, in a case where the number of items ordered by the user is less than or equal to a predetermined value (S1203: YES), the control unit 200 extracts a recipe whose “timing” is a main dish among recipes extracted in Step S1102 (S1204).

On the other hand, in a case where the number of items ordered by the user is not less than or equal to the predetermined value (S1203: NO), the control unit 200 determines whether or not there is a recipe including an ingredient preferred by the user among the corresponding recipes (S1205). In a case where it is determined that there is no recipe including an ingredient preferred by the user (S1205: NO), the control unit 200 determines whether or not the number of candidate recipes is plural (S1207).

On the other hand, in a case where it is determined that there is a recipe including an ingredient preferred by the user (S1205: YES), the control unit 200 extracts a recipe including an ingredient preferred by the user among the corresponding recipes (S1206), and determines whether or not the number of extracted recipes is plural (S1207). In a case where it is not determined in Step S1207 that the number of candidate recipes is plural (in a case where the number of candidate recipes is one) (S1207: NO), the control unit 200 replaces an ingredient that is not eatable by the user among ingredients of the candidate recipe with a substitute ingredient (S1208) and determines the recipe (S1209).

On the other hand, in a case where it is determined that the number of candidate recipes is plural (S1207: YES), the control unit 200 excludes a recipe including an ingredient that is not eatable by the user among the candidate recipes (S1210). Next, in a case where it is determined that the number of candidate recipes is not plural (S1211: NO), the control unit 200 determines the recipe (S1208) and ends the operation. On the other hand, in a case where the number of candidate recipes is plural (S1211: YES), the control unit 200 calculates a nutrient intake amount of the user on the basis of the number of items ordered by the user, determines, on the basis of the calculated intake amount and an ideal nutrient amount of the user, a recipe containing a necessary nutrient as much as possible (S1212), and ends the operation.

The above-described flow of the operation related to recipe determination performed by the control unit 200 with an emphasis on a preference is merely an example, and is not limited to such an example.

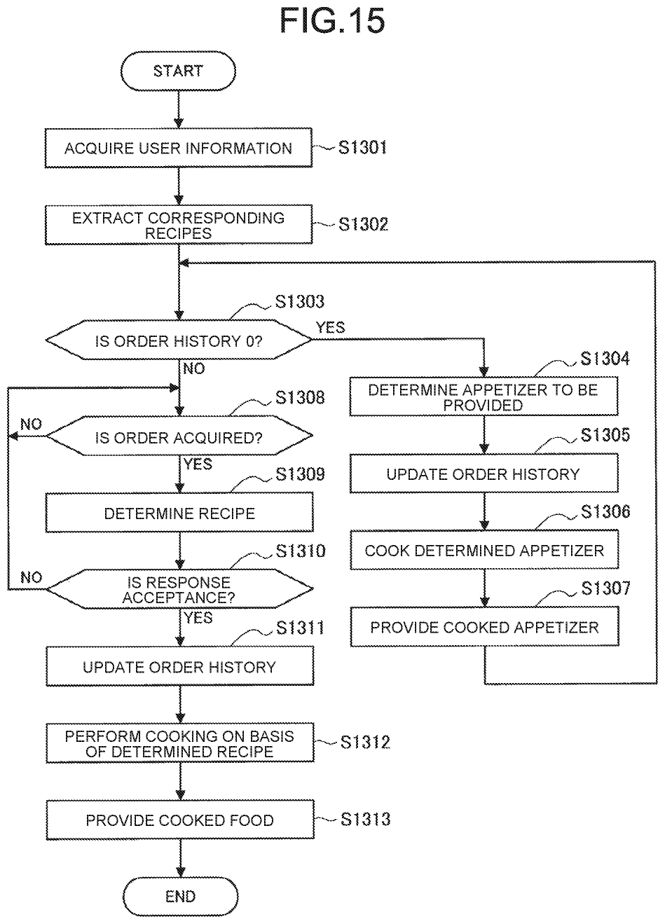

Further, the control unit 200 can determine a recipe on the further basis of the number of food orders, recommend the recipe to the user, and provide the food on the basis of a response from the user. FIG. 15 is a diagram for describing an operation related to recipe determination further based on the number of food orders and provision of a food based on a response to a recipe recommendation by the cooking system according to the present embodiment.

Referring to FIG. 15, first, the control unit 200 acquires an order condition from the input unit 100 (S1301). Next, the control unit 200 extracts recipes corresponding to the order condition from the food information DB 410 on the basis of the order condition acquired in Step S1101 (S1302).

Next, in a case where the number of orders in an order history is 0 (S1303: YES), the control unit 200 determines an appetizer to be provided (S1304). Next, the control unit 200 performs update so as to add the number of orders in the order history of the user in the storage unit 400 by one (S1305). Next, the cooking unit 500 cooks the appetizer determined in Step S1304 (S1306). Next, the appetizer cooked in Step S1306 is provided by a robot or the like (S1307), and the process returns to Step S1303.

On the other hand, in a case where the number of orders in the order history is not 0 (S1303: NO) and the control unit 200 does not acquire a food order (S1308: NO), the process returns to Step S1308. Further, in a case where the number of orders in the order history is not 0 (S1303: NO) and the control unit 200 acquires a food order (S1308: YES), the control unit 200 determines a recipe on the basis of the food information DB 410, the ingredient information DB 420, and the user information DB 430 (S1309).

Next, the control unit 200 recommends the recipe determined in Step S1309 to the user, and determines whether or not a response from the user is acceptance (S1310). In a case where it is determined that the response is not acceptance (S1310: NO), the process returns to Step S1308. On the other hand, in a case where it is determined that the response is acceptance (S1310: YES), the control unit 200 performs update so as to add the number of orders in the order history of the user in the storage unit 400 by one (S1311). Next, the cooking unit 500 performs cooking on the basis of the recipe determined in Step S1309 (S1312). Finally, the appetizer cooked in Step S1306 is provided by a robot or the like (S1313), and the cooking system ends the operation.

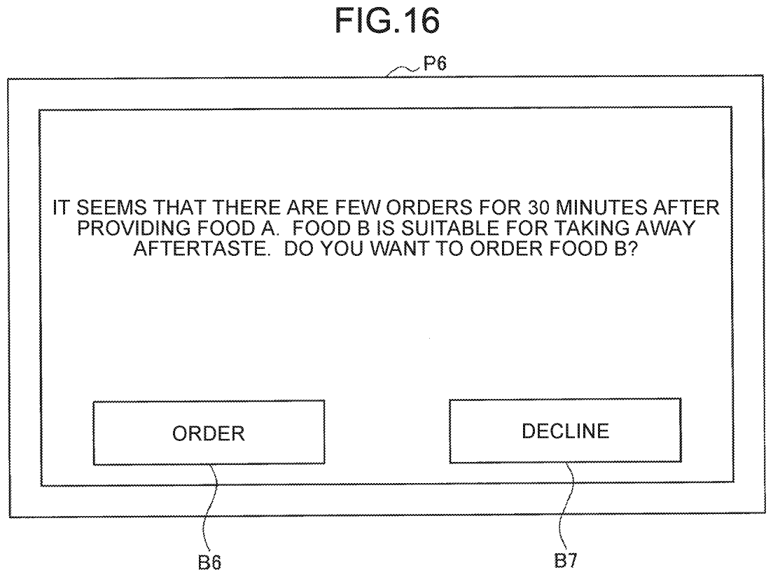

The flow of the operation of the control unit 200 that is related to the recipe recommendation and the order history has been described above. Here, the control unit 200 may recommend a recipe in a case where the number of orders from the user is less than or equal to a predetermined value for a predetermined time. FIG. 16 is a diagram for describing a recipe recommendation by the control unit 200 in a case where the number of orders from the user is less than or equal to a predetermined value for a predetermined time according to the present embodiment.

FIG. 16 illustrates a display screen P6 related to a recipe recommendation. In FIG. 16, the control unit 200 determines a recipe on the basis of the fact that the number of orders from the user is less than or equal to a predetermined value for a predetermined time. The control unit 200 recommends the determined recipe by causing the output unit 600 to display the determined recipe to the user. Meanwhile, the user responds to the food recommendation by touching any of buttons shown in the display screen P6.

In the example of FIG. 16, the control unit 200 recommends, to the user, a food B that is determined as being most suitable for taking away the aftertaste of a food A, on the basis of the fact that an order for the food A is included in the order history. Here, the control unit 200 determines whether or not the food B is ordered on the basis of a touch on any of buttons B6 and B7, the touch being acquired by the input unit 100 and performed by the user. For example, in a case where the user touches the button B6 on which “order” is written, the control unit 200 causes the cooking unit 500 to cook the food B. Note that an example of the display performed by the output unit 600 is not limited to such an example. The input unit 100 may receive an utterance of the user as a response to the recipe recommendation.

In this way, in a case where there are few food orders from the user for a predetermined time, it is possible to obtain more realistic user information. With such a function, it becomes possible to determine a recipe that is more suitable for each user.

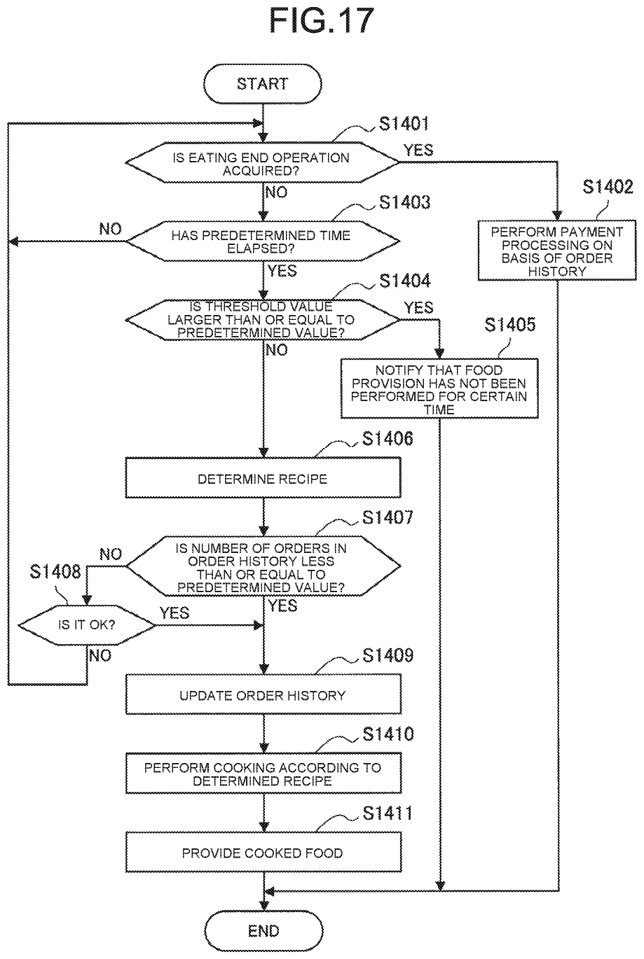

Next, a flow of an operation related to a case where there is no food order from the user for a predetermined time according to the present embodiment will be described, the operation being performed by the control unit 200. FIG. 17 is a diagram for describing the flow of the operation related to a case where there is no order from the user for a predetermined time according to the present embodiment, the operation being performed by the control unit 200.

Referring to FIG. 17, first, in a case where the input unit 100 has acquired an eating end operation performed by the user (S1401: YES), the control unit 200 performs payment processing on the basis of the order history stored in the storage unit 400 (S1402) and ends the operation. On the other hand, in a case where the input unit 100 has not acquired the eating end operation performed by the user (S1401: NO), the control unit 200 determines whether or not a predetermined time has elapsed from the most recent food order from the user (S1403). On the other hand, in a case where it is determined that the predetermined time has not elapsed from the most recent food order from the user (S1403: NO), the process returns to Step S1401.

On the other hand, in a case where it is determined that the predetermined time has elapsed from the most recent food order from the user (S1403: YES), the control unit 200 determines whether or not the predetermined time elapsed from the most recent food order from the user is longer than or equal to a threshold value (S1404). In a case where it is determined that the predetermined time is longer than or equal to the threshold value (S1404: YES), the control unit 200 causes the output unit 600 to notify the user that food provision to the user has not been performed for a certain time (S1405), and ends the operation.

On the other hand, in a case where it is determined that the predetermined time is not longer than or equal to the threshold value (S1404: NO), the control unit 200 determines a recipe on the basis of the food information DB 410, the ingredient information DB 420, and the user information DB 430 (S1406). Next, in a case where the number of orders in the order history is less than or equal to a predetermined value (S1407: YES), the control unit 200 performs an update so as to add the recipe determined in Step S1406 to the order history of the user (S1409). On the other hand, in a case where the number of orders in the order history is not less than or equal to the predetermined value (S1407: NO), the control unit 200 recommends the recipe determined in Step S1406 to the user, and determines whether or not a response from the user to the recommendation is acceptance (S1408). In a case where it is determined that the response is not acceptance (S1408: NO), the process returns to Step S1401. On the other hand, in a case where it is determined that the response is acceptance (S1408: YES), the control unit 200 performs an update so as to add the recipe determined in Step S1406 to the order history of the user (S1409). After performing Step S1409, the control unit 200 causes the cooking unit 500 to cook the recipe determined in Step S1406 (S1410). Finally, the food cooked in Step S1406 is provided to the user by a person or a robot (S1411), and the control unit 200 ends the operation.

In a case where a cooking robot cooks a determined recipe, the user may not be able to recognize that the cooked food includes an ingredient that is not preferred by the user. For example, in a case where the user orders a food having a food name from which it is difficult to guess ingredients, it is not possible to determine whether or not the provided food includes an ingredient to which the user has an allergy.

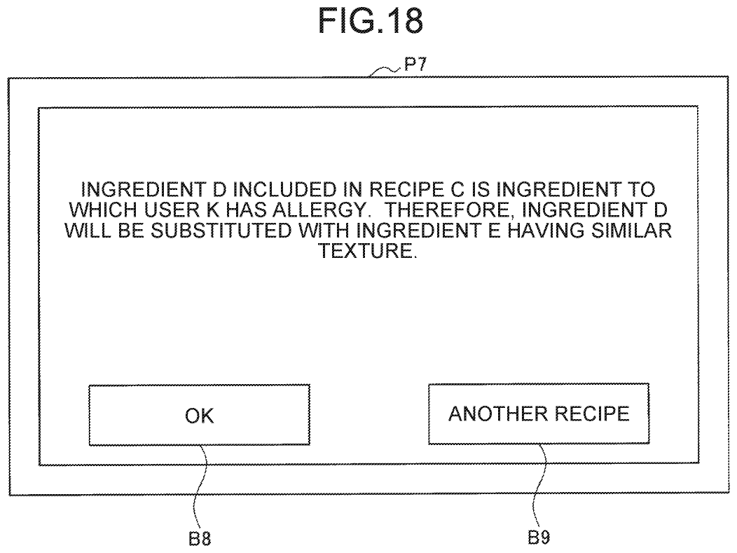

Therefore, the control unit 200 may notify the user of ingredient substitution. FIG. 18 is a diagram for describing an example of notification related to ingredient substitution according to the present embodiment, the notification being performed by the control unit 200. FIG. 18 illustrates a display screen P7 for notification of ingredient substitution. In FIG. 18, the user selects whether to accept ingredient substitution or to change to another recipe, for a predetermined ingredient of a recipe determined by the control unit 200.

In the example of FIG. 18, the control unit 200 causes the output unit 600 to provide notification of substitution of an ingredient D included in a recipe C with an ingredient E on the basis of a touch on any of buttons B8 and B9, the touch being acquired by the input unit 100 and performed by the user. Here, in a case where the user touches the button B8 on which “OK” is written, the control unit 200 determines a recipe in which the ingredient D included in the recipe C is substituted with the ingredient E.

As such, it is possible to determine a recipe that is more suitable for the user's situation by notifying the user of ingredient substitution. Further, it becomes possible for a user who has an allergy to a predetermined ingredient to eat foods with an easy mind by notifying the user of ingredient substitution as described above.

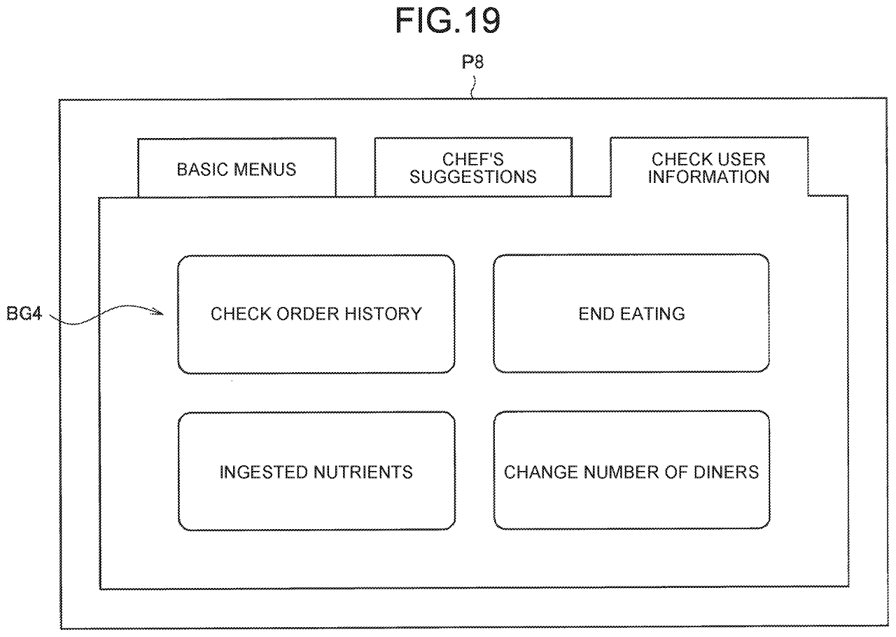

Subsequently, a display control related to user information according to the present embodiment will be described, the display control being performed by the control unit 200. FIG. 19 is a diagram for describing the display control related to user information according to the present embodiment, the display control being performed by the control unit 200. FIG. 19 illustrates a menu display screen P8 related to the display of user information. Further, a button group BG4 related to the display of user information is shown in the menu display screen P8. The control unit 200 causes the output unit 600 to output information regarding a user on the basis of a touch on the button group BG4 related to the display of user information, the touch being acquired by the input unit 100 and performed by the user.

In the example of FIG. 19, the control unit 200 causes the output unit 600 to display the order history in a case where, for example, the input unit 100 acquires a touch on an “check order history” button. As the control unit 200 performs the display control related to user information as described above, the user can eat foods while checking and changing his/her situation. With such a function, it is possible to improve convenience for the user.

Hereinabove, an example of a situation where the user eats alone has been described. However, people usually eat together with an accompanying person at a restaurant or the like. Here, the control unit 200 according to the present embodiment may determine a recipe or cause the cooking unit 500 to perform cooking in consideration of the presence of an accompanying person.

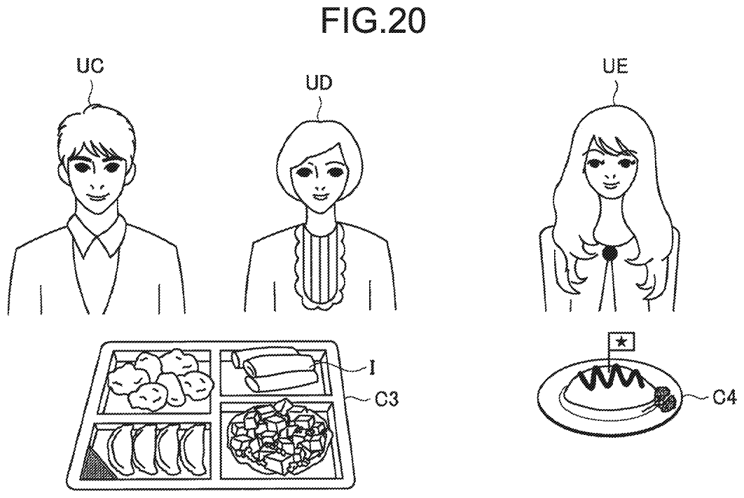

FIG. 20 is an example for describing an example of recipe determination performed by the control unit 200 in a case where a user to be provided with a food has an accompanying person according to the present embodiment. In FIG. 20, the control unit 200 may determine a recipe on the further basis of user information regarding at least one accompanying user of a user to be provided with a food. Specifically, the control unit 200 may determine a recipe on the further basis of, for example, a result of comparison between user information regarding the user to be provided with a food and the user information regarding the accompanying user.

The example of FIG. 20 will be described below. Users UC, UD, and UE are illustrated. The users UC, UD, and UE ordered a food C3 to be shared (a menu to be shared). Here, it is assumed that the food C3 includes an ingredient I, and that the user information DB 430 stores information indicating that the user UE has an allergy to the ingredient I. At this time, the control unit 200 determines a recipe of the food C3 to be served on a shared plate for the users UC and UD, and determines a recipe of another food C4 that does not include the ingredient I for the user UE. Here, the cooking unit 500 puts the food C4 on a plate other than that of the food C3.

In this way, it is possible to determine a recipe suitable for each user even in a situation where there is an accompanying person, that is, a plurality of users eat together.

### 2. HARDWARE CONFIGURATION EXAMPLE

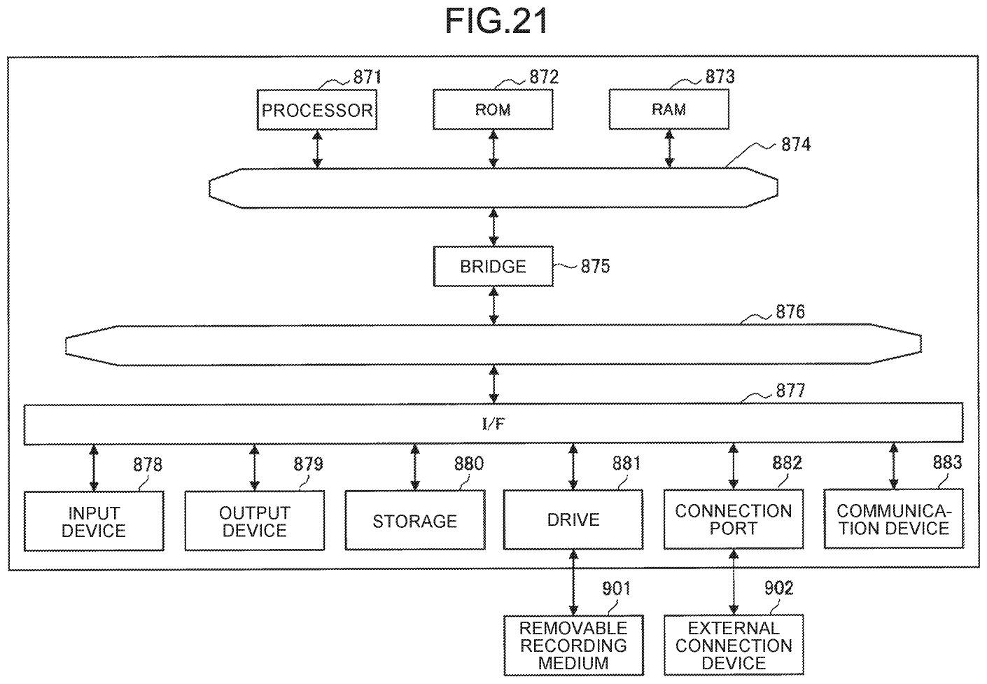

Next, a hardware configuration example of the information processing apparatus including the control unit 200 according to an embodiment of the present disclosure will be described. FIG. 21 is a block diagram illustrating the hardware configuration example of the information processing apparatus including the control unit 200 according to an embodiment of the present disclosure. Referring to FIG. 21, an information processing terminal 10 and an information processing server 20 include, for example, a processor 871, a read only memory (ROM) 872, a random access memory (RAM) 873, a host bus 874, a bridge 875, an external bus 876, an interface 877, an input device 878, an output device 879, a storage 880, a drive 881, a connection port 882, and a communication device 883. Note that the hardware configuration illustrated herein is an example, and some of the components may be omitted. In addition, components other than the components illustrated herein may be further included.

(Processor 871)

For example, the processor 871 functions as an arithmetic processing device or a control device, and controls an overall operation of each component or a part thereof on the basis of various programs recorded in the ROM 872, the RAM 873, the storage 880, or a removable recording medium 901.

(ROM 872 and RAM 873)

The ROM 872 is means that stores a program read by the processor 871, data to be used for calculation, or the like. The RAM 873 temporarily or permanently stores, for example, the program read by the processor 871, various parameters that change as appropriate when the program is executed, or the like.

(Host Bus 874, Bridge 875, External Bus 876, and Interface 877)

The processor 871, the ROM 872, and the RAM 873 are mutually connected via, for example, the host bus 874 capable of high-speed data transmission. Meanwhile, the host bus 874 is connected to the external bus 876, which has a relatively low data transmission rate, via the bridge 875, for example. In addition, the external bus 876 is connected to various components via the interface 877.

(Input Device 878)

As the input device 878, for example, a mouse, a keyboard, a touch panel, a button, a switch, a lever, or the like is used. Further, a remote controller capable of transmitting a control signal using infrared rays or other radio waves may also be used as the input device 878. In addition, the input device 878 includes a speech input device such as a microphone.

(Output Device 879)

The output device 879 is a device capable of visually or audibly notifying a user of acquired information, such as a display device such as a cathode ray tube (CRT), a liquid crystal display (LCD), or an organic EL, an audio output device such as a speaker or a headphone, a printer, a mobile phone, or a facsimile. In addition, the output device 879 according to the present disclosure includes various vibration devices capable of outputting haptic stimulation.

(Storage 880)

The storage 880 is a device for storing various types of data. As the storage 880, for example, a magnetic storage device such as a hard disk drive (HDD), a semiconductor storage device, an optical storage device, or a magneto-optical storage device is used.

(Drive 881)

The drive 881 is a device that reads information recorded on the removable recording medium 901 such as a magnetic disk, an optical disk, a magneto-optical disk, or a semiconductor memory, or writes information to the removable recording medium 901.

(Removable Recording Medium 901)

The removable recording medium 901 is, for example, a DVD medium, a Blu-ray (registered trademark) medium, an HD DVD medium, or various semiconductor storage media. It is a matter of course that the removable recording medium 901 may be, for example, an IC card equipped with a non-contact IC chip, or an electronic device.

(Connection Port 882)

The connection port 882 is a port for connecting an external connection device 902 such as a universal serial bus (USB) port, an IEEE 1394 port, a small computer system interface (SCSI), an RS-232C port, or an optical audio terminal.

(External Connection Device 902)

The external connection device 902 is, for example, a printer, a portable music player, a digital camera, a digital video camera, or an IC recorder.

(Communication Device 883)

The communication device 883 is a communication device for connection to a network, and is, for example, a communication card for a wired or wireless LAN, Bluetooth (registered trademark), or a wireless USB (WUSB), a router for optical communication, a router for an asymmetric digital subscriber line (ADSL), or a modem for various communications.

### 3. CONCLUSION

As described above, the cooking system according to the embodiment of the present disclosure can determine a recipe that is more suitable for each user, and thus can determine a recipe that is more suitable for each user even in a case where the avoidance factor is difficult to quantitatively determine.

As described above, the preferred embodiments of the present disclosure have been described in detail with reference to the accompanying drawings, but the technical scope of the present disclosure is not limited to such examples. It will be apparent to those skilled in the art to which the present disclosure pertains that various modifications or alterations can be conceived within the scope of the technical idea described in the claims and it is naturally understood that these modifications or alterations fall within the technical scope of the present disclosure.

Furthermore, the effects described in the present specification are merely illustrative or exemplary and are not restrictive. That is, the technology according to the present disclosure can exhibit, in addition to or in place of the above-described effects, other effects obvious to those skilled in the art from the description of the present specification.

In addition, the respective steps related to the processing performed by the information processing terminal 10 and the information processing server 20 in the present specification are not necessarily performed in a time-series manner in the order described in the sequence diagram. For example, the respective steps related to the processing performed by the information processing terminal 10 and the information processing server 20 may be performed in an order different from the order described in the flowchart, or may be performed in parallel.

Note that the following configurations also fall within the technical scope of the present disclosure.

(1) An information processing apparatus comprising:

a control unit that determines a recipe on a basis of user information including an avoidance level of a user for a predetermined ingredient, food information regarding each food, and ingredient information including information regarding each ingredient, wherein the avoidance level for the predetermined ingredient is set on a basis of an avoidance factor of the user for the predetermined ingredient, and the food information includes information indicating the ingredient used to cook the food.

(2) The information processing apparatus according to (1), wherein

the control unit determines the recipe in which the ingredient is substituted so that the avoidance level of the ingredient is lower than or equal to a predetermined level.

(3) The information processing apparatus according to (2), wherein

the avoidance factor includes a fact that the user has an allergy to the predetermined ingredient, and

the control unit determines the recipe that does not include the ingredient to which the user has an allergy so that the avoidance level of the ingredient is lower than or equal to the predetermined level.

(4) The information processing apparatus according to (2) or (3), wherein

the avoidance factor includes a preference of the user for the predetermined ingredient, and

the control unit determines the recipe so that the avoidance level for the ingredient is lower than or equal to the predetermined level, on a basis of the preference of the user for the predetermined ingredient.

(5) The information processing apparatus according to (4), wherein

the control unit determines the recipe including an ingredient that the user prefers more than other ingredients, or the recipe that that does not include an ingredient that the user dislikes more than the other ingredients, so that the avoidance level of the ingredient is lower than or equal to the predetermined level.

(6) The information processing apparatus according to any one of (1) to (5), wherein

the control unit determines the recipe on a further basis of a food order history of the user.

(7) The information processing apparatus according to (6), wherein

the food order history of the user includes at least one order condition, and

the control unit determines the recipe on a further basis of the at least one order condition.

(8) The information processing apparatus according to (7), wherein

the order condition includes the avoidance factor, and

the control unit determines the recipe on a further basis of the avoidance level set on a basis of the avoidance factor included in the order condition.

(9) The information processing apparatus according to any one of (6) to (8), wherein

the food order history of the user includes nutritional information regarding a food ordered by the user,

the ingredient information includes information indicating the nutritional information of a nutrient contained in each ingredient, and

the control unit determines the recipe on a further basis of the nutritional information.

(10) The information processing apparatus according to (9), wherein

the control unit determines, on a basis of an ideal nutrient intake amount of the user and an amount of a nutrient contained in a food provided to the user, the recipe so that a nutrient intake amount of the user reaches the ideal nutrient intake amount.

(11) The information processing apparatus according to any one of (6) to (10), wherein

the control unit determines the recipe on a basis of the food order history of the user in a case where it is determined that the number of new orders is less than or equal to a predetermined value for a predetermined time after a predetermined order from the user, on a basis of orders included in the food order history of the user.

(12) The information processing apparatus according to any one of (1) to (11), wherein

the user information is updated on a basis of a feedback on the provided food from the user, and

the control unit determines the recipe on a basis of the updated user information.

(13) The information processing apparatus according to any one of (1) to (12), wherein

the control unit determines the recipe on a further basis of the user information regarding other users.

(14) The information processing apparatus according to (13), wherein

the control unit determines the recipe on a basis of the user information regarding another user, who is similar to a user to be provided with the food, among the other users.

(15) The information processing apparatus according to (13) or (14), wherein

the control unit estimates the avoidance factor of the user on a further basis of the user information regarding the other users, and determines the recipe on a further basis of the estimated avoidance factor.

(16) The information processing apparatus according to (14), wherein

the control unit determines the recipe on a further basis of the user information regarding at least one accompanying user of the user to be provided with the food.

(17) The information processing apparatus according to (16), wherein

the control unit determines the recipe on a further basis of a result of comparison between the user information regarding the user to be provided with the food and the user information regarding the accompanying user.

(18) The information processing apparatus according to any one of (1) to (17), wherein

the control unit causes a cooking robot to cook the food corresponding to the determined recipe.

(19) An information processing method comprising:

determining, by a processor, a recipe on a basis of user information including an avoidance level of a user for a predetermined ingredient, food information regarding cooking of each food, and ingredient information including information regarding each ingredient, wherein

the avoidance level for the predetermined ingredient is set on a basis of an avoidance factor of the user for the predetermined ingredient, and

the food information includes information indicating the ingredient used to cook the food.

(20) A program for causing a computer to function as an information processing apparatus including

a control unit that determines a recipe on a basis of user information including an avoidance level of a user for a predetermined ingredient, food information regarding cooking of each food, and ingredient information including information regarding each ingredient, wherein

the avoidance level for the predetermined ingredient is set on a basis of an avoidance factor of the user for the predetermined ingredient, and

the food information includes information indicating the ingredient used to cook the food.

### REFERENCE SIGNS LIST

100 INPUT UNIT
200 CONTROL UNIT
400 STORAGE UNIT
500 COOKING UNIT
600 OUTPUT UNIT

1. An information processing apparatus comprising:
a control unit that determines a recipe on a basis of user information including an avoidance level of a user for a predetermined ingredient, food information regarding each food, and ingredient information including information regarding each ingredient, wherein
the avoidance level for the predetermined ingredient is set on a basis of an avoidance factor of the user for the predetermined ingredient, and
the food information includes information indicating the ingredient used to cook the food.

2. The information processing apparatus according to claim 1, wherein
the control unit determines the recipe in which the ingredient is substituted so that the avoidance level of the ingredient is lower than or equal to a predetermined level.

3. The information processing apparatus according to claim 2, wherein
the avoidance factor includes a fact that the user has an allergy to the predetermined ingredient, and
the control unit determines the recipe that does not include the ingredient to which the user has an allergy so that the avoidance level of the ingredient is lower than or equal to the predetermined level.

4. The information processing apparatus according to claim 2, wherein
the avoidance factor includes a preference of the user for the predetermined ingredient, and
the control unit determines the recipe so that the avoidance level for the ingredient is lower than or equal to the predetermined level, on a basis of the preference of the user for the predetermined ingredient.

5. The information processing apparatus according to claim 4, wherein
the control unit determines the recipe including an ingredient that the user prefers more than other ingredients, or the recipe that that does not include an ingredient that the user dislikes more than the other ingredients, so that the avoidance level of the ingredient is lower than or equal to the predetermined level.

6. The information processing apparatus according to claim 1, wherein
the control unit determines the recipe on a further basis of a food order history of the user.

7. The information processing apparatus according to claim 6, wherein
the food order history of the user includes at least one order condition, and
the control unit determines the recipe on a further basis of the at least one order condition.

8. The information processing apparatus according to claim 7, wherein
the order condition includes the avoidance factor, and
the control unit determines the recipe on a further basis of the avoidance level set on a basis of the avoidance factor included in the order condition.

9. The information processing apparatus according to claim 6, wherein
the food order history of the user includes nutritional information regarding a food ordered by the user,
the ingredient information includes information indicating the nutritional information of a nutrient contained in each ingredient, and
the control unit determines the recipe on a further basis of the nutritional information.

10. The information processing apparatus according to claim 9, wherein
the control unit determines, on a basis of an ideal nutrient intake amount of the user and an amount of a nutrient contained in a food provided to the user, the recipe so that a nutrient intake amount of the user reaches the ideal nutrient intake amount.

11. The information processing apparatus according to claim 6, wherein
the control unit determines the recipe on a basis of the food order history of the user in a case where it is determined that the number of new orders is less than or equal to a predetermined value for a predetermined time after a predetermined order from the user, on a basis of orders included in the food order history of the user.

12. The information processing apparatus according to claim 1, wherein
the user information is updated on a basis of a feedback on the provided food from the user, and
the control unit determines the recipe on a basis of the updated user information.

13. The information processing apparatus according to claim 1, wherein
the control unit determines the recipe on a further basis of the user information regarding other users.

14. The information processing apparatus according to claim 13, wherein
the control unit determines the recipe on a basis of the user information regarding another user, who is similar to a user to be provided with the food, among the other users.

15. The information processing apparatus according to claim 13, wherein
the control unit estimates the avoidance factor of the user on a further basis of the user information regarding the other users, and determines the recipe on a further basis of the estimated avoidance factor.

16. The information processing apparatus according to claim 14, wherein
the control unit determines the recipe on a further basis of the user information regarding at least one accompanying user of the user to be provided with the food.

17. The information processing apparatus according to claim 16, wherein
the control unit determines the recipe on a further basis of a result of comparison between the user information regarding the user to be provided with the food and the user information regarding the accompanying user.

18. The information processing apparatus according to claim 1, wherein
the control unit causes a cooking robot to cook the food corresponding to the determined recipe.

19. An information processing method comprising:
determining, by a processor, a recipe on a basis of user information including an avoidance level of a user for a predetermined ingredient, food information regarding cooking of each food, and ingredient information including information regarding each ingredient, wherein
the avoidance level for the predetermined ingredient is set on a basis of an avoidance factor of the user for the predetermined ingredient, and
the food information includes information indicating the ingredient used to cook the food.

20. A program for causing a computer to function as an information processing apparatus including
a control unit that determines a recipe on a basis of user information including an avoidance level of a user for a predetermined ingredient, food information regarding cooking of each food, and ingredient information including information regarding each ingredient, wherein
the avoidance level for the predetermined ingredient is set on a basis of an avoidance factor of the user for the predetermined ingredient, and
the food information includes information indicating the ingredient used to cook the food.
